(19) United States
(12) Patent Application Publication
(10) Pub. No.: US 2016/0296438 A1
(43) Pub. Date: Oct. 13, 2016

(54) PROTECTIVE ACTION OF LUTEIN AGAINST BLUE LIGHT ON HUMAN SKIN CELL LINES

(71) Applicant: Kemin Industries, Inc., Des Moines, IA (US)

(72) Inventors: Naresh Modepalli, West Des Moines, IA (US); Satish Nayak, West Des Moines, IA (US)

(21) Appl. No.: 15/094,278

(22) Filed: Apr. 8, 2016

Related U.S. Application Data
(60) Provisional application No. 62/144,409, filed on Apr. 8, 2015.

(51) Int. Cl. A61K 8/34; A61Q 19/08; A61Q 17/04

(52) U.S. Cl. CPC A61K 8/345; A61Q 17/04; A61Q 19/08

(57) ABSTRACT

Human skin can be protected against damage upon exposure to blue light by application of an effective amount of lutein to the skin.

### RELATED APPLICATIONS

This application claims priority to U.S. Patent Application Ser. No. 62/144,409, filed Apr. 8, 2015, and incorporated herein in its entirety by this reference.

### BACKGROUND OF THE INVENTION

The present invention relates generally to protecting human skin against light damage and, more specifically, to the use of lutein to protect human skin from damage by exposure to blue light.

The electromagnetic energy in the solar spectrum that reaches earth surface consists of UV radiation, visible light and infrared radiation. The effect of light and radiation on skin and tissues has been studied extensively in the past especially the use of light and radiation irradiation sources for treating pathological dermatology conditions (Frain-Bell W. The effect of light on the skin. British Journal of Dermatology. 1988, 119:479-485). Light travels as photons and the absorption of photons by a molecular structure in the physiological system initiates the photobiological reactions (Rupert C. S. The biological effectiveness of Ultraviolet light. National cancer institute monograph. 1978, 50:85-89. ISSN: 0083-1921).

The deleterious effects of skin exposure to ultraviolet (UV) radiation (both UVA and UVB) are well known. The UV radiation is categorized into three regions, UVC, UVB and UVA. Most of the UVC is filtered by ozone layer and high energy low wavelength UVB get absorbed in the upper layers of skin, i.e. epidermal region, while UVA penetrates little deeper into dermal regions of the skin. So far, most of the studies on skin exposure to light were concentrated on the role of UV irradiation due to its high energy, photo reactivity and its associated damage to the skin while the role of visible light has been less extensively investigated (Mahmoud B. H., Hexsel C. L., Hamzavi I. H. and Lim H. W. Effects of Visible Light on the Skin. Photochem Photobiol, 2008, 84: 450-462). The effect of visible light on skin in terms of cytokine release, matrix metalloproteinase (MMPs) production was investigated by Liebel et al and few others, but the effects of blue light on skin was not explored extensively (Liebel F, Kaur S, Ruvolo E, Kollias N and Southall M. D. Irradiation of Skin with Visible Light Induces Reactive Oxygen Species and Matrix-Degrading Enzymes. Journal of Investigative Dermatology. 2012. 132: 1901-1907; doi:10.1038/jid.2011.476).

Visible light is the region of light with 400-700 nm in the electromagnetic spectrum. Blue light is the portion of the electromagnetic spectrum in the visible region with wavelengths ranging from 400-500 nm. The wavelengths of blue light are close to UVA spectrum (315-400 nm) and the blue region of the visible spectrum is particularly important because it has a relatively high energy and longer wavelengths that can penetrate tissue deeper than UV light due to its longer wavelengths (Godley B. F., Shamsi F. A., Liang F. Q., Jarrett S. G., Davies S. and Boulton M., Blue light induces mitochondrial DNA damage and free radical production in epithelial cells, J. Biol. Chem. 280 (2005) 21061-21066; Opländer C., Hidding S., Frauke B., Werners F. B., Born M., PalluaN. And Suschek C. V. Effects of blue light irradiation on human dermal fibroblasts. Journal of Photochemistry and photobiology B: Biology. 2011. 103: 118-125).

The adverse effects of blue light on a variety of cell lines were studied in the past by various research groups and a few of them were briefly summarized by Oplander et al. Especially, a lot of focus was given to the penetration of blue light in retinal cells and its effect on the generation of age related macular degeneration was studied by many research groups. Only a few studies were focused on the toxic effects of blue light on skin and related tissues.

The purpose of this study was to investigate the effect of blue light on cell viability, proliferation and generation of reactive oxygen species in human skin keratinocytes (HEK) and Human dermal fibroblast cells (HDF) and to identify the protective effect of Kemin's FloraGLO™ Lutein against blue light. Using cell viability and reactive oxygen species generation assays, the reduction of cell toxicity when FloraGLO Lutein was used against blue light was investigated in HDF and HEK cells.

### SUMMARY OF THE INVENTION

Skin undergoes stress induced by exposure to visible and ultraviolet radiations (UV) from sun. As a body's natural response to environmental stress, skin cells release a vast array of cytokines and proteolytic enzymes such as matrix metalloproteinase (MMPs), which lead to the breakdown of collagen and other extracellular matrix proteins, ultimately leading to appearance of fine lines and wrinkles.

Blue light contains wavelengths of the visible spectrum at 400-500 nm which are close to UVA spectrum. The blue region (400-500 nm) of the visible spectrum is particularly important because it has a relatively high energy and longer wavelengths that can penetrate tissue deeper than UV light. Cell viability/proliferation studies and reactive oxygen species assays were performed on human skin keratinocytes and fibroblasts cells to determine the toxicity of blue light and the protective action of lutein against blue light. From the cell culture studies, blue light induces toxicity in these cell lines and lutein was able to provide a protective effect.

### BRIEF DESCRIPTION OF THE FIGURES

FIG. 1 is a chart of the reactive oxygen species (ROS) assay scheme.

FIG. 2 is a chart of the cell viability shown as % cell viability for different treatments in response to exposure of blue light in human epidermal keratinocytes. “No treatment” controls are without exposure to blue light at room temperature in the biological hood and at 37° C. in the CO₂ incubator. The data points represented are the average of n=3±S.D. (Means with same letters are not statistically significant from each other).

FIG. 3 is a chart of the cell viability shown as % cell viability for different treatments in response to exposure of blue light in human dermal fibroblasts. “No treatment” controls are without exposure to blue light at room temperature in the biological hood and at 37° C. in the CO₂ incubator. The data points represented are the average of n=3±S.D. (Means with same letters are not statistically significant from each other).

FIGS. 4a and 4b are standard calibration curves for 2′, 7′-dichlorodihydrofluorescin (DCF) assay in (a) keratinocytes and (b) fibroblasts media; DCF prepared from 0-10000 nM (x-axis) was plotted against the relative fluorescence units (y-axis).

FIG. 5 is a chart of the results of an ROS assay using human epidermal keratinocytes for different treatments in response to exposure of blue light; the negative control is without exposure to blue light at room temperature; the data points represented are the average of n=3±S.D.; cells treated with pure DMSO and exposed to blue light serves as a control for DMSO effect. (Means with same letters are not statistically significant from each other).

FIG. 6 is a chart of the results of an ROS assay using human dermal fibroblasts for different treatments in response to exposure of blue light; the negative control is without exposure to blue light at room temperature; the data points represented are the average of n=3±S.D.; cells treated with pure DMSO and exposed to blue light serves as a control for DMSO effect. (Means with same letters are not statistically significant from each other).

### DETAILED DESCRIPTION OF THE PREFERRED EMBODIMENTS

This disclosure relates to cosmetic and/or pharmaceutical preparations that contain lutein. Illustrative cosmetic compositions include, for example, sunscreen compositions, blue light and/or ultraviolet radiation protective compositions, anti-aging compositions, anti-wrinkle compositions, moisturizer compositions, skin soothing compositions, skin softening compositions, skin treating compositions, anti-inflammatory compositions, antioxidant compositions, and free radical inhibitive compositions.

Cosmetic and/or pharmaceutical preparations based on lutein show surprisingly good skin and care and protecting properties against stress and against environmental influences coupled with high dermatological compatibility. The preparations are also distinguished by a high antioxidation capacity that, on the one hand, protects the skin against inflammatory reactions and against oxidation-induced skin aging processes; on the other hand, cosmetic preparations are simultaneously protected against oxidative degradation (deterioration). In addition, the products thus obtained are capable of preventing damage to human fibroblasts and keratinocytes by blue light and may therefore be used as sun protection factors in cosmetics. The preparations are useful in antioxidative stress applications and also the repair of damaged skin (both blue light and normal damaged).

The quantity of lutein used in the preparations mentioned is governed by the concentration of the individual ingredients and by the way in which the extracts are used. In general, lutein is used in a quantity based on the final cosmetic and/or pharmaceutical preparation, for example, an amount of from about 10 ppm to about 1% by weight, preferably about 0.25 ppm to about 0.1% by weight, and more particularly about 50 ppm to about 500 ppm by weight. The preparations may be produced by conventional processes known in the art.

This disclosure also relates to the use of lutein in skin and/or hair care preparations, particularly against stress; in moisture-regulating moisturizers. The preparations are particularly useful in antioxidative stress applications and also the repair of damaged skin and hair (both blue light and normal damaged).

Care preparations in the context of this disclosure are understood to be skin care preparations. These care preparations have blue light protection properties. In principle, the extracts according to this disclosure may be used in a variety of cosmetic products.

The moisturizing system of the compositions can be formulated to provide substantial moisturizing to the skin, including simultaneously with the protection of the skin. It has been found that substantial moisturizing to the skin can be achieved by the moisturizing system of this disclosure.

Suitable optional moisturizing components include, but are not limited to, one or more polyols, siloxanes, naturally occurring fats and oils, or any combinations thereof.

The one or more polyols that may be used as moisturizing components include, but are not limited to, glycerin, propylene glycol, butylene glycol, hexylene glycol, pentylene glycol, caprylyl glycol, sorbitol, or any combinations thereof.

The one or more siloxanes that may be used as moisturizing components in this disclosure include, but are not limited to, dimethicone, cyclomethicone, phenyl trimethicone, phenyl dimethicone, cetyl dimethicone, stearyl dimethicone, amodimethicone, C₃₀₋₄₅ alkyl dimethicone, C₃₀₋₄₅ alkyl methicone, cetearyl methicone, dimethicone copolyol, cyclopentasiloxane, or any combinations thereof

The one or more naturally occurring fats and oils that may be used as moisturizing components in this disclosure include, but are not limited to, shea butter, shea butter oil, cocoa butter, jojoba butter, aloe butter, olive butter, coconut oil, jojoba oil, olive oil, sunflower seed oil, meadowfoam seed oil, macadamia nut oil, sesame oil, borage seed oil, or any combinations thereof

Suitable UV filters may include, but are not limited to, dibenzoylmethane, oxybenzone, sulisobenzone, dioxybenzone, menthyl anthranilate, para aminobenzoic acid (PABA) ester, benzophenone-3, butyldibenzoylmethane, dimethyl cinnamate, octyl methoxycinnamate, DEA methoxycinnamate, octocrylene, drometrizole trisiloxane, octyl salicylate, homomenthyl salicylate, octyl dimethyl PABA, TEA salicylate, 4-methyl benzylidene camphor, 3-benzylidene camphor, benzylidene camphor sulfonic acid ester, octyl triazone, phenyl benzimidazole sulfonic acid ester, terephthalydiene dicamphor sulfonic acid ester, di-t-butyl hydroxybenzylidene camphor, ethyl PABA, butylmethoxy dibenzoylmethane (avobenzone), terephthalydiene methylene bis-benzotriazoyltetramethylbutyl-phenol, diethylhexyl-2,6-naphthalate, bis-ethylhexyloxyphenol methoxyphenol triazine, hydroxy methylphenyl benzotriazole, methylene bis-benzotriazoyltetramethylbutylphenol, bis-ethylhexyloxyphenol methoxyphenol triazine, hydroxybenzophenone, a benzotriazole, a dibenzoyl methane, an oxanilide, a hydroxy cinnamate, oil dispersible titanium dioxide, oil dispersible zinc oxide, a silicone-anchored sunscreen, para aminobenzoic acid (PABA), salicylic acid, TEA salicylate, benzylidene camphor sulfonic acid, phenyl benzimidazole sulfonic acid, terephthalydiene dicamphor sulfonic acid, hydroxy cinnamic acid, any derivatives thereof, or any combinations thereof.

Other suitable additives may include, but are not limited to, antioxidants, such as, erythrobic acid, sodium metabisulfite, sodium sulfite, rosemary extract, tocopherol, a derivative of tocopherol including a tocotriene, carotene, a carotenoid, lutein or lutein ester, a carotenoid, a phenolic antioxidant, a bioflavonoid, a plant extract, or any combinations thereof; keratolytic agents, such as, salicylic acid, resorcinol, peroxide of an organic acid, or any combinations thereof; anti-inflammatory agents, such as, steroidal and non-steroidal anti-inflammatory agents, plant extracts that have demonstrated anti-inflammatory activity, or any combinations thereof; vitamins, such as, Vitamin K, Vitamin C, retinol (vitamin A), tocopherol, or any combinations thereof; emollients, such as, cetearyl octanoate, octyl palmitate, glyceryl monostearate, caprylic trigylceride, capric trigylceride, or any combinations thereof; humectants, such as, hyaluronic acid, one or more derivatives of hyaluronic acid, or any combinations thereof; skin penetration enhancers, such as, ozone, SEPA, butylene glycol, cis-isomer of an unsaturated fatty acid, or any combinations thereof; emulsifiers, such as, glyceryl stearate, cetearyl alcohol, cetyl alcohol, PEG-40 stearate, or any combinations thereof; thickening agents, such as, xanthan gum, carbomer, clay, hydroxyethyl cellulose, or any combinations thereof; preservatives, such as, an alkyl paraben, an alcohol, salts of benzoic acid, salts of sorbic acid, or any combinations thereof; colorants, such as, synthetic and natural colorants, henna, caramel, photo-chromic and thermo-chromic colorants and pigments, surface-treated or hydrophobically modified colorants, or any combinations thereof; organic acids and their derivatives, such as citric acid, glycolic acid, glutamic acid, gluco delta lactone, or any combinations thereof; chelating agents, such as, disodium EDTA; pH adjusters, such as, an acid, a base, a buffer, or any combinations thereof, to adjust and maintain the pH to about 3.0 to about 7.5; fragrances; proteins; peptides; and amino acids.

The composition is applicable to a variety of personal care product forms including, but not limited to body wash, bar soap, liquid soap, lather, skin and/or hair care preparation, cream, foam, gel, lotion, solution, emulsion, pomade, mousse, balm, stick, pump spray, aerosol spray, or any combinations thereof

This disclosure provides a method of simultaneously protecting the skin against blue light and moisturizing skin having the step of topically applying to the skin an effective amount of a composition having lutein and one or more moisturizing systems.

This disclosure also relates to the use of lutein in sun protection preparations, e.g., sunscreens. Sun protection factors or blue light protection factors in the context of this disclosure are light protection factors that are useful in protecting human skin and/or hair against harmful effects of direct and indirect solar radiation.

The compositions containing lutein are used as blue light filters that convert blue light radiation into harmless heat. They may additionally be present in combination with other sun protection factors or UV protection factors.

These UV protection factors are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances: 3-benzylidene camphor or 3-benzylidene norcamphor and derivatives thereof, for example 3-(4-methylbenzylidene)-camphor as described in EP-B 1 0693471; 4-aminobenzoic acid derivatives, preferably 4-(dimethylamino)-benzo-ic acid-2-ethylhexyl ester, 4-(dimethylamino)-benzoic acid-2-octyl ester and 4-(dimethylamino)-benzoic acid amyl ester; esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (octocrylene); esters of salicylic acid, preferably salicylic acid-2-ethylhexyl ester, salicylic acid-4-isopropylbenzyl ester, salicylic acid homomenthyl ester; derivatives of benzophenone, preferably 2-hydroxy-4-methoxybenzophe-none, 2-hydroxy-4-methoxy-4′-methylbenzophenone, 2,2′-dihydroxy-4-methoxyb-enzophenone; esters of benzalmalonic acid, preferably 4-methoxybenzmalonic acid di-2-ethylhexyl ester; triazine derivatives such as, for example, 2,4,6-trianilino-(p-carb-o-2′-ethyl-1′-hexyloxy)-1,3,5-triazine and octyl triazone or dioctyl butamido triazone; propane-1,3-diones such as, for example, 1-(4-tert.butylphenyl)-3-(-4′-methoxyphenyl)-propane-1,3-dione; ketotricyclo(5.2.1.0)decane derivatives.

For sunscreen preparations, the compositions containing lutein may be present in an amount effective to impart a sunscreen booster effect in the sunscreen composition and can increase the effectiveness of conventional sunscreen preparations in protecting human skin and/or hair against harmful effects of direct and indirect solar radiation.

This disclosure also relates to the use of compositions containing lutein in preparations against fibroblast and/or keratinocyte damage by blue light radiation and as anti-inflammatory additives.

Moisture-regulating moisturizers according to this disclosure are understood to be skin care preparations that are intended to regulate skin moisture. In the context of this disclosure, this conforms to the definition of a moisturizer. They are substances or mixtures of substances which provide cosmetic and/or pharmaceutical preparations with the ability to reduce the release of moisture from the stratum corneum (horny layer) after application to and spreading over the surface of the skin.

The compositions according to this disclosure may be used as skin soothing and/or skin softener additives for cosmetic and/or pharmaceutical preparations used in skin care.

In principle, the compositions according to this disclosure may be used as anti-inflammatory additives for any cosmetic and/or pharmaceutical preparations used against inflammation of the skin and hence in skin care. The inflammation of the skin may be caused by various factors.

In an embodiment, cosmetic compositions are provided to be topically applied to the skin. The cosmetic compositions of this disclosure can be formulated for topical administration and applied to the skin so as to reduce oxidative stress, e.g., compositions having antioxidant properties that have the ability to terminate free radical chain reactions in biological systems. Oxidative stress is a result of an imbalance between antioxidative defense systems and the formation of reactive oxygen species including free radicals. Oxidative stress can damage DNA, proteins, lipids and carbohydrates and may also alter intracellular signaling processes. The damage can contribute to cell injury and death, accelerate the aging process, and promote many diseases, such as cancer, cardiovascular diseases, and Parkinson's disease. The preparations are particularly useful in antioxidative stress applications and also the repair of damaged skin and hair (both blue light and normal damaged).

This disclosure also relates to the use of compositions containing lutein as antioxidants or radical traps. Antioxidants are capable of inhibiting or preventing changes caused by the effects of oxygen and other oxidative processes in the substances to be protected. The effect of antioxidants consists mainly in their acting as radical traps for the free radicals occurring during autoxidation. The compositions exhibit desirable free radical scavenging activity.

The cosmetic compositions of this disclosure are typically used in topical form. The topical form can be a solution, emulsion, serum, skin and/or hair cleanser, body wash, body scrub, bar soap, liquid soap, shampoo lather, deodorant, skin and/or hair care preparation, foam, mousse, cream, lotion, pomade, balm, stick, gel, pump spray, aerosol spray, and combinations thereof. In an illustrative embodiment, the cosmetic compositions of this disclosure are used in foams in personal care applications such as soaps, shampoos, skin cleansers, bubble bath, shaving soaps, oral products, and the like. The cosmetic compositions can impart desired foaming, emulsifying, cleansing, dispersing, and/or skin soothing properties.

The compositions of this disclosure comprise a “cosmetically acceptable carrier” to act as a diluent, dispersant or carrier for the ingredients, so as to facilitate its distribution and uptake when the composition is applied to the skin. Vehicles other than or in addition to water can include liquid or solid emollients, solvents, humectants, thickeners, powders, and perfumes.

In the context of this disclosure, biogenic agents are, for example, tocopherol, tocopherol acetate, tocopherol palmitate, ascorbic acid, deoxyribonucleic acid, retinol, bisabolol, allantoin, phytantriol, panthenol, amino acids, ceramides, pseudoceramides, essential oils, other plant extracts and vitamin complexes. The biogenic agents may be present in the compositions of this disclosure in quantities of normally about 0.01 to about 20% by weight, preferably about 0.5 to about 15% by weight, and more preferably about 0.5 to about 10% by weight. The biogenic agents are conventional materials known in the art.

Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides. The water soluble additives may be present in the compositions of this disclosure in quantities of normally about 0.01 to about 20% by weight, preferably about 0.5 to about 15% by weight, and more preferably about 0.5 to about 10% by weight. The water-soluble additives are conventional materials known in the art.

Suitable preservatives are, for example, phenoxyethanol, formaldehyde solution, parabens, pentanediol or sorbic acid. The preservatives are conventional materials known in the art.

It has been found that the compositions of the present invention have strong antioxidant properties which have the capability to terminate free radical chain reactions in biological systems and therefore may provide additional health benefits to consumers. Severe oxidative stress, a result of an imbalance between anti oxidative defense systems and the formation of reactive oxygen species including free radicals, can damage DNA, proteins, lipids and carbohydrate and may also alter intracellular signaling processes. The damage could contribute to cell injury and death, accelerate the aging process, and promote many diseases, such as cancer, cardiovascular diseases, diabetes, arthritis, Alzheimer's disease, Parkinson's disease, and free radical related diseases.

The compositions of this disclosure can be utilized in many cosmetic applications. Preferred cosmetic applications include, for example, the following:

(i) sunscreen lotions; since compositions have the capability to protect skin blue light radiation and can be used to make sun protecting lotions;

(ii) anti-aging or anti wrinkling creams; the compositions have natural antioxidant properties in addition to having the capacity to scavenge radicals causing damage to human cells; these properties are known to decelerate the aging process; and

(iii) moisturizers; the compositions have the potential to protect the skin from the water loss which ultimately reduces the risks of many skin diseases.

### Example 1

### Materials and Methods

Cell culture studies. All the cell culture studies were performed using HEK cells [HEK001 (ATCC® CRL-2404™) lot #61331463] continuous cell lines and Human Skin Fibroblast (HDF) [CCD1093Sk (ATCC® CRL2115™) lot #3296816] cell lines [ATCC, Manassas, Va.].

The growth medium used for HEK cells was prepared according to ATCC guidelines. Growth media consisting of keratinocyte serum free medium (GIBCO-BRL 17005042, lot #1638561) with 5 ng/mL human recombinant epidermal growth factor (EGF) (ATCC, lot # 1584416) and 2 mM L-glutamine (ATCC, lot #62195752) (without bovine pituitary extract and serum) and supplemented with penicillin (10000 units) and streptomycin (10 mg/mL) solution (Gibco, lot #1469707). The growth medium used for HDF cells consisting of eagle's Minimum Essential Medium (EMEM) (ATCC-302003, lot #62028896) with 10% FBS and supplemented with penicillin (10000 units) and streptomycin (10 mg/mL) solution. Cells were grown in cell culture flasks (75 cm²) [Fisher Scientific, catalog number 10-126-37] to approximately 80% confluence in 5% CO₂ environment incubator (NuAire, MN) maintained at 37° C. Confluent cells were passed into subsequent cultures by trypsinization following neutralization and centrifugation. HEK and HDF cultures were routinely observed visually at the beginning and the end of every experiment and after media changes and splitting. All experiments were performed with HEK cells at passage no. 97 and HDF cells at passage no. 9.

Cell viability and proliferation assays. The effect of blue light on keratinocytes and fibroblasts cells was investigated with the help of MTT (3-(4,5-Dimethylthiazol-2-yl)-2,5-Diphenyltetrazolium Bromide), MTS (3-(4.5-dimethylthiazol--2-yl)-5-(3-carboxymethoxyphenyl)-2-(4-sulfophenyl)-2H-tetrazolium) and CellTiter-Glo® luminescent cell viability assays by measuring viability and proliferation of metabolically active cells. After reaching confluence, cells were detached from cell culture flasks and seeded into clear/black/white wall 96-well microplates (Fisher Scientific, catalogue number 07-200-87) at a density of 100,000 cells/mL (200 μL). Cell count was performed using hemacytometer (Hausser scientific, PA) after staining a 10 μl aliquot of cell suspension with 10 μl of Trypan blue stain (0.4%) [Life Technologies, Grand Island, N.Y.].

FloraGLO™ Lutein (production number M080033, lot number 1405102477, manufactured date Jun. 04, 2014) was solubilized in sterile dimethyl sulfoxide (DMSO) [ATCC, lot #61908420] with intermittent shaking for at least 6 hours to get a stock of 2 mg/mL solution. From the stock; 2.5, 5 and 10 μl of the lutein solution was added to wells containing 100 μl media to get a final lutein concentration of 50, 100 and 200 ppm in corresponding wells. Hydrogen peroxide (H₂O₂) [Sigma, lot #WKBS8306V] solution at 1 mM was served as a positive control in all the studies. Pure DMSO at 2.5, 5 and 10 μl was added to few wells to have the DMSO control.

After adding the treatments and all controls the microplates were exposed to blue LED lights (476 nms, 1900 lux) [Item No #LED72-B(QL-72B); Mainlandmart] for 9 hours from a distance of 30 cm. Cells that were not treated with lutein served as blue light control. Another microplate prepared in similar way which was not exposed to blue light served as no treatment control. At the end of 9 hour treatment period, the cell viability assays were performed.

MTS assay. The CellTiter 96® AQueous one solution reagent (Promega, Madison, Wis., catalog number G3580, lot #0000120696) was completely thawed in water bath at 37° C. for 10 minutes before use and 20 μl of the this reagent was added to each well in the 96 well plate containing the cells and the plate was incubated at 37° C. in a humidified, 5% CO₂ atmosphere for 4 hours. After incubation the absorbance at 490 nm was recorded using microplate Reader (SpectraMax® Me5, Molecular Devices, LLC. Sunnyvale, Calif.).

MTT assay. The CellTiter 96® non-radioactive cell proliferation assay (Promega, Madison, Wis., catalog number G4002, lot #0000130044) is based on the cellular conversion of a tetrazolium salt into a formazan product. A premixed optimized dye solution (15 μl) from CellTiter 96® assay was added to culture wells of a 96-well plate and incubated for 4 hours. Living cells convert the tetrazolium component of the dye solution into a formazan product. Solubilization solution/stop mix (100 μl) was added to the culture wells to solubilize the formazan product, and the absorbance was measured at 570 nm.

The CellTiter-Glo® Luminescent Cell Viability Assay. The CellTiter-Glo® Luminescent Cell Viability Assay (Promega, Madison, Wis., catalog number G755A, lot #0000104605) is a homogeneous method to determine the number of viable cells in culture based on quantitation of the ATP present, which signals the presence of metabolically active cells. The homogeneous assay procedure involves adding a single reagent (CellTiter-Glo® Reagent) directly to cells cultured in serum-supplemented medium. 100 μl of Cell Titer-Glo reagent was added to each well containing cells with 100 μl base medium. After stabilization luminescence was recorded. The homogeneous “add-mix-measure” format results in cell lysis and generation of a luminescent signal proportional to the amount of ATP present. The amount of ATP is directly proportional to the number of cells present in culture. The CellTiter-Glo® assay relies on the properties of a proprietary thermostable luciferase (Ultra-Glo™ Recombinant Luciferase), which generates a stable “glow-type” luminescent signal.

Shielding of blue light. Cells were grown to confluence as described earlier. In order to observe the shielding capabilities of FloraGLO Lutein against blue light, microplates seeded with HEK and HDF cells were covered with another flat bottom sterile plate on top containing FloraGLO™ Lutein prepared at 50, 100 and 200 ppm concentrations, using dimethyl sulfoxide (DMSO). These cells were exposed to blue LED lights for 9 hours and uncovered microplates (direct exposure of blue light) containing the cells served as blue light control. Cell seeded in a microplate kept away from blue light served as no treatment control. The blue light source was positioned above the culture plate. After 9 hours of exposure, the cell viability was measured using a colorimetric assay (MTS assay) in which the tetrazolium compound is bioreduced by living cells to produce formazan.

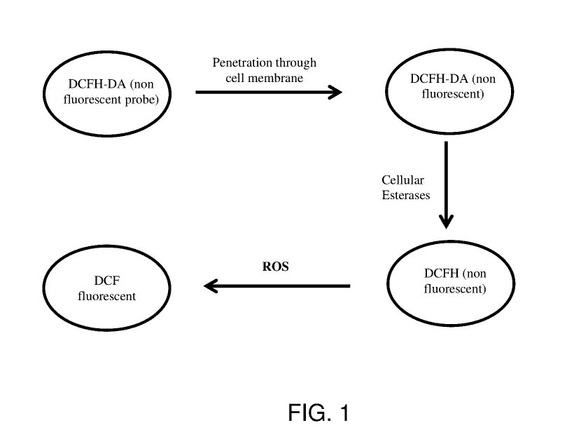

Reactive Oxygen Species (ROS) assay. A chart of the reactive oxygen species (ROS) assay scheme is shown in FIG. 1. ROS activity was measured using Oxiselect™ intracellular ROS assay kit (Cell BioLabs, San Diego, Calif., catalogue number STA 342, lot #59342025). The cells were seeded into 96-well microplates at a density of 100,000 cells/mL (200 μL). Both the cell lines were treated with cell permeable fluorogenic probe 2′, 7′-dichlorodihydrofluorescin diacetate (DCFH-DA). Cells were washed with D-PBS for couple of times and treated with lutein at different concentrations and exposed to blue LED lights (476 nm) for 9 hours (Cells that were not treated with lutein will serve as blue light control). Cells treated with pure DMSO serves as a control for DMSO effect in presence and absence of blue light. Cells were washed again 2-3 times with D-PBS and 100 μL of fresh medium and 100 μL of 2× cell lysis buffer was added. After 5 minutes incubation, 150 μL of this medium was transferred to a black wall 96-well plate and fluorescence was measured. Hydrogen peroxide at 1 mM was used as positive control.

Statistical analysis. Values were reported as means±standard deviations (SD). Data were analyzed using an unpaired t-test. P<0.05 was considered to be significant.

### Results

FloraGLO™ Lutein was solubilized in DMSO and applied to cell cultures. Lutein, due to its lipophilic nature, does not dissolve in cell culture compatible media. Due to limited solubility of lutein in DMSO the volume of DMSO used to achieve 50-200 ppm final concentration of lutein in cell culture well was high. In our earlier attempts for cell viability assays with various detection mechanisms, this high concentrations of DMSO might be interacting with cell culture membranes and causing a reduction in cell proliferation and decrease in cell viability compared to controls.

With the limited success in cell viability studies due to presence of high concentrations of DMSO in the cell culture studies, an experiment was designed to prevent the penetration of blue light by blocking it with lutein solution by placing the lutein solution on top of the microplate containing the cells as a shield.

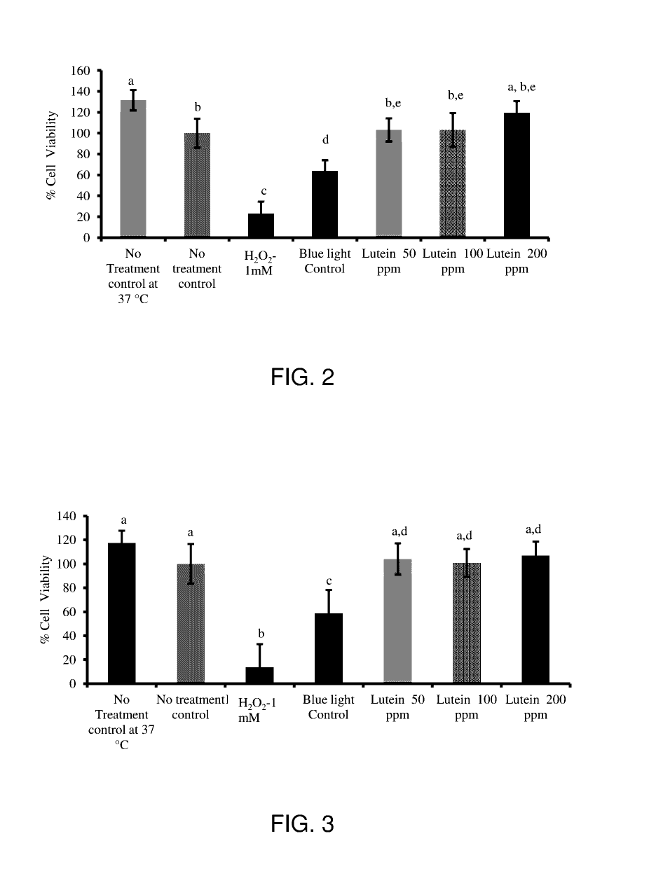

Cell proliferation assay from blue light shielding studies with Lutein. The results of the cell viability assay with shielding of blue light in HEK and HDF cells are presented in FIGS. 2 and 3. The study was performed using MTS assay. There was a significant reduction in cell proliferation (˜52% in case of HEK cells from FIGS. 2 and ˜55% in case of HDF cells from FIG. 3) in the case of blue light exposure compared to no treatment control (p<0.05). Likewise, in case of cells exposed to blue light in presence of lutein at 50,100 and 200 ppm concentrations, the cell proliferation was significantly improved compared to cells with blue light control studies (p<0.05) suggesting that lutein was able to prevent the damage caused by blue light in both the cell lines.

ROS assay. Intracellular reactive oxygen species (ROS) activity and the oxidative stress was measured using Oxiselect™ intracellular ROS assay kit which can measure hydroxyl, peroxyl and other free radical in the cells. Reactive oxygen species can cause oxidative stress at cellular level and oxidative stress can activate NF-KB signaling pathway, stress-activated kinases, and leads to cell death by necrosis.

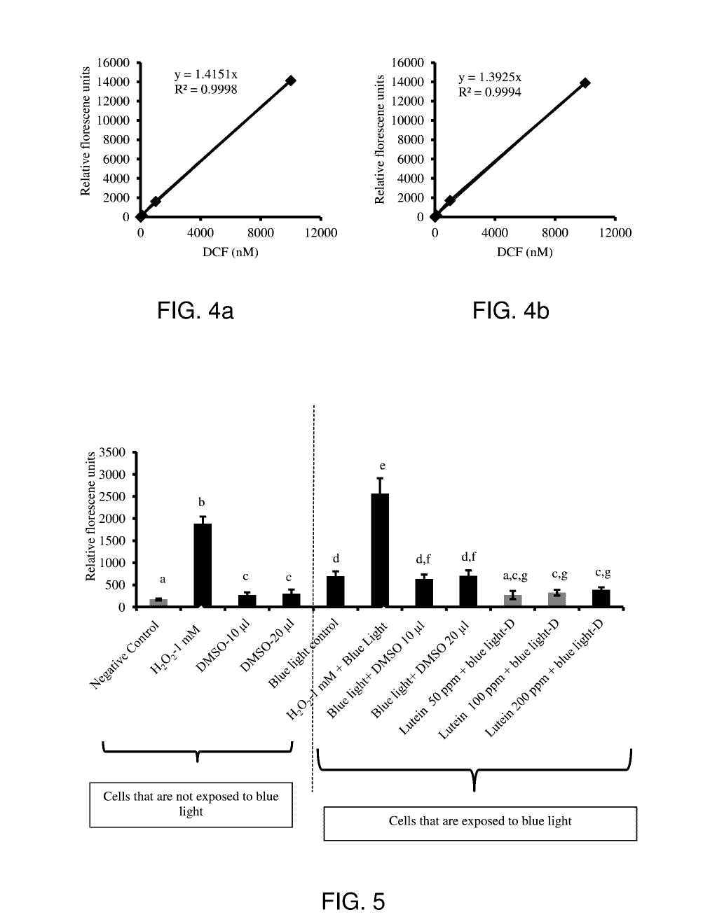

Upon addition to cells, the non-fluorescent DCFH-DA permeates well into across the cell membrane and rapidly deacetylated by cellular esterases to a non-fluorescent 2′, 7′-dichlorodihydrofluorescin (DCFH). DCFH will be rapidly oxidized to fluorescent 2′, 7′-dichlorodihydrofluorescin (DCF-green fluorescence) in presence of reactive oxygen species. The fluorescence intensity is proportional to the ROS levels within the cell cytosol. A standard calibration curve was plotted with different concentrations (0 nM, 0.01 nM, 0.1 nM, 1 nM, 10 nM, 100 nM, 1000 nM and 10,000 nM) of standard DCF in both of the cell media using relative fluorescence units (RFU) (FIGS. 4a and 4b).

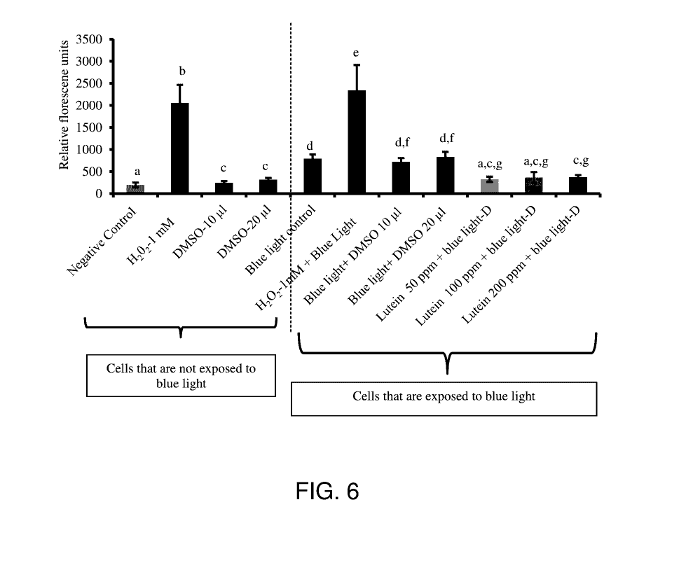

The generation of reactive oxygen species in both HEK and HDF cells in terms of relative fluorescence units is shown in FIGS. 5 and 6. When cells were exposed to blue light, there was a significant increase in the fluorescence signals which indicates the presence of DCF with in the cells generated by ROS. When cells were exposed to blue light in presence of lutein at 50, 100 and 200 ppm concentrations, the florescence signal was low, confirming that lutein absorbs the blue light and results in protection from blue light by preventing the generation of ROS. DMSO control studies were also performed in presence and absence of blue light by having pure DMSO at 10 μl and 20 μl volumes to identify any possible interference of DMSO in ROS production.

### Discussion

After exposure to blue light for 9 hours there was ˜50-55% reduction in cell proliferation compared to no treatment control. H₂O₂ used at 1 mM served as a positive control to induce cytotoxicity in these cells. From FIGS. 2 and 3, the lutein solution prepared at 50, 10 and 200 ppm concentrations shielded the blue light effectively as can be seen in the % cell proliferation compared against just blue light exposure. The results indicate that lutein prevents the exposure of blue light to keratinocytes and fibroblast cells by absorbing the blue light at all tested concentrations. Being a bioactive, lutein has the potential benefits to prevent skin damages and premature signs of aging by blocking harmful blue light rays that induce cytotoxicity in HEK and HDF cells. From ROS studies it is clearly evident that the presence of lutein in cell culture studies can prevent the generation of reactive oxygen species even when exposed to blue light and lutein can protect the cells from oxidative stress that can be caused by blue light exposure.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

We claim:

1. A method of protecting human skin against damage upon exposure to blue light, comprising the step of applying a composition containing an effective amount of lutein to the skin.

2. The method of claim 1, wherein the lutein prevents the exposure of blue light to keratinocytes and fibroblast cells by absorbing the blue light.

3. A method of protecting human skin premature signs of aging, comprising the step of applying a composition containing an effective amount of lutein to the skin to block blue light rays that thereby reducing the induction of cytotoxicity in HEK and HDF cells.

4. A method of reducing the generation of reactive oxygen species in humans when exposed to blue light, comprising the step of applying a composition containing an effective amount of lutein to the skin.
